(19) United States
(12) Patent Application Publication
(10) Pub. No.: US 2016/0287106 A1
(43) Pub. Date: Oct. 6, 2016

(54) METHOD FOR ASSESSING PATIENT RISK FOR VENTRICULAR TACHYCARDIA

(71) Applicant: Xerox Corporation, Norwalk, CT (US)

(72) Inventors: Lalit Keshav MESTHA, Fairport, NY (US); Luisa Fernanda POLANIA-CABRERA, Webster, NY (US)

(21) Appl. No.: 14/674,868

(22) Filed: Mar. 31, 2015

(51) Int. Cl. A61B 5/024; A61B 5/00

(52) U.S. Cl. CPC A61B 5/02416; A61B 5/7264; A61B 5/7225; A61B 5/7282

(57) ABSTRACT

What is disclosed is a system and method for assessing risk for ventricular tachycardia from a time-series signal of a patient monitored for cardiac function assessment. One embodiment involves receiving a time-series signal which contains frequency components that relate to the function of the subject's heart. Signal segments of interest are identified in the time-series signal. Time-domain features are extracted for each signal segment of interest. The time-domain features are arranged into a two dimensional feature vector. Each feature vector is associated with a respective signal segment. A magnitude of each signal segment's respective feature vector is determined. Signal segments are classified as being ventricular premature contraction based on each segment's associated magnitude. If at least three consecutive signal segments or at least three consecutive portions of the same signal segment have been classified as ventricular premature contraction, then the subject's risk of a ventricular tachycardia is assessed as elevated.

### TECHNICAL FIELD

The present invention is directed to systems and methods for using a time-series signal containing frequency components which relate to patient cardiac function to assess that patient's risk for ventricular tachycardia.

### BACKGROUND

Methods for early detection of ventricular tachycardia are increasingly needed to increase patient survival rates. Therefore, what is needed are systems and methods for using a time-series signal to assess patient risk for ventricular tachycardia.

### INCORPORATED REFERENCES

The following U.S. Patents, U.S. Patent Applications, and Publications are incorporated herein in their entirety by reference.
“Classifying A Time-Series Signal As Ventricular Premature Contraction”, U.S. patent application Ser. No. 14/674,736, by Polanía-Cabrera et al., (Attorney Docket: 20141525US01 (420-P0239)).
“Classifying A Time-Series Signal As Ventricular Premature Contraction And Ventricular Tachycardia”, U.S. patent application Ser. No. 14/674,791, by Polanía-Cabrera et al., (Attorney Docket: 20150063US01 (420-P0240)).
“Identifying A Type Of Cardiac Event From A Cardiac Signal Segment”, U.S. patent application Ser. No. 14/492,948, by Xu et al.
“System And Method For Detecting An Arrhythmic Cardiac Event From A Cardiac Signal”, U.S. patent application Ser. No. 14/519,607, by Kyal et al.
“Determining Cardiac Arrhythmia From A Video Of A Subject Being Monitored For Cardiac Function”, U.S. patent application Ser. No. 14/245,405, by Mestha et al.
“Method And Apparatus For Monitoring A Subject For Atrial Fibrillation”, U.S. patent application Ser. No. 13/937,740, by Mestha et al.
“Continuous Cardiac Signal Generation From A Video Of A Subject Being Monitored For Cardiac Function”, U.S. patent application Ser. No. 13/871,766, by Kyal et al.
“Continuous Cardiac Pulse Rate Estimation From Multi-Channel Source Video Data With Mid-Point Stitching”, U.S. patent application Ser. No. 13/871,728, by Kyal et al.
“Determining Cardiac Arrhythmia From A Video Of A Subject Being Monitored For Cardiac Function”, U.S. patent application Ser. No. 13/532,128, by Mestha et al.
“Continuous Cardiac Pulse Rate Estimation From Multi-Channel Source Video Data”, U.S. patent application Ser. No. 13/528,307, by Kyal et al.
“Estimating Cardiac Pulse Recovery From Multi-Channel Source Data Via Constrained Source Separation”, U.S. patent application Ser. No. 13/247,683, by Mestha et al.

### BRIEF SUMMARY

What is disclosed is a system and method for using a time-series signal to assess patient risk for ventricular tachycardia. One embodiment hereof involves the following. A time-series signal is received which contains frequency components that relate to the function of the subject's heart. Signal segments of interest are identified in the time-series signal. Time-domain features comprising the peak-to-peak interval between cardiac pulses and pulse amplitudes are extracted for each signal segment of interest. The time-domain features are arranged into a two dimensional feature vector. Each feature vector is associated with a respective signal segment. A magnitude of each signal segment's respective feature vector is determined. In a manner more fully disclosed herein, signal segments are classified as being ventricular premature contraction based on each segment's associated magnitude. If at least three consecutive signal segments or at least three consecutive portions of the same signal segment have been classified as ventricular premature contraction, then the subject's risk of a ventricular tachycardia is assessed as being elevated.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 is a flow diagram which illustrates one example embodiment of the present method for assessing patient risk for ventricular tachycardia in accordance with the methods disclosed herein;

FIG. 2 is a continuation of the flow diagram of FIG. 1 with flow processing continuing with respect to node A; and

FIG. 3 is a block diagram of one example signal processing system for assessing patient risk for ventricular tachycardia as described with respect to the flow diagrams of FIGS. 1 and 2.

### DETAILED DESCRIPTION

What is disclosed is a system and method for using a time-series signal to assess patient risk for ventricular tachycardia.

### NON-LIMITING DEFINITIONS

“Plethysmography” is the study of relative blood volume changes in blood vessels which reside beneath the surface of skin tissue.

A “photoplethysmographic (PPG) signal” is a signal obtained using an optical instrument which captures the blood volume pulse over time.

A “videoplethysmographic (VPG) signal” is a signal extracted from processing batches of image frames of a video of the skin surface.

A “subject” refers to a living being. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

“Cardiac function” refers to the function of the heart and, to a larger extent, to the cardio-vascular system. Cardiac function can be impacted by a variety of factors including age, stress, disease, overall health, and by environmental conditions such as altitude and pressure.

“Ventricular Tachycardia” refers to an abnormal heart rate or abnormal heart rhythm. Tachycardias range from slow to fast heart rates. Some ventricular tachycardias are only slightly abnormal. The teachings disclosed herein facilitate diagnosis of ventricular tachycardia.

A “time-series signal” is a signal which contains frequency components which relate to cardiac function. The time-series signal can be a photoplethysmographic (PPG) signal or a videoplethysmographic (VPG) signal. Methods for obtaining time-series signals are disclosed in several of the incorporated references by Kyal et al and Mestha et al. One or more signal segments of interest are identified in the time-series signal.

A “signal segment of interest” refers to a portion of a time-series signal which has been identified as being of interest. Methods for obtaining a segment of a signal are well established in the signal processing arts. One method for classifying signal segments based on a clustering method is disclosed in the incorporated reference entitled: “Identifying A Type Of Cardiac Event From A Cardiac Signal Segment”, by Xu et al. Signal segments have a fixed length. A length of a signal segment can comprise of any of: a single cardiac cycle, a normalized cardiac cycle, multiple cardiac cycles, and multiple normalized cardiac cycles. Time-domain features are extracted from the signal segments of interest.

“Time-domain features” refers to the peak-to-peak interval between cardiac pulses in the signal segment and the cardiac pulse amplitude. Time-domain features are arranged into feature vectors. Methods for generating vectors are well established in the mathematical arts.

“Receiving a time-series signal” is intended to be widely construed and includes: retrieving, capturing, acquiring, or otherwise obtaining time-series signals for processing in accordance with the teachings hereof. Time-series signals can also be retrieved from a memory or storage device of the device used to capture those signals, or from a media such as a CDROM or DVD, retrieved from a remote device over a network, or downloaded from a web-based system or application which makes such signals available for processing.

It should be appreciated that the steps of “determining”, “analyzing”, “identifying”, “receiving”, “processing”, “classifying”, “extracting” “selecting”, “performing”, “detrending”, “filtering”, smoothing”, and the like, as used herein, include the application of any of a variety of signal processing techniques as well as mathematical operations according to any specific context or for any specific purpose. It should be appreciated that such steps may be facilitated or otherwise effectuated by a microprocessor executing machine readable program instructions such that an intended functionality can be effectively performed.

### Example Flow Diagram

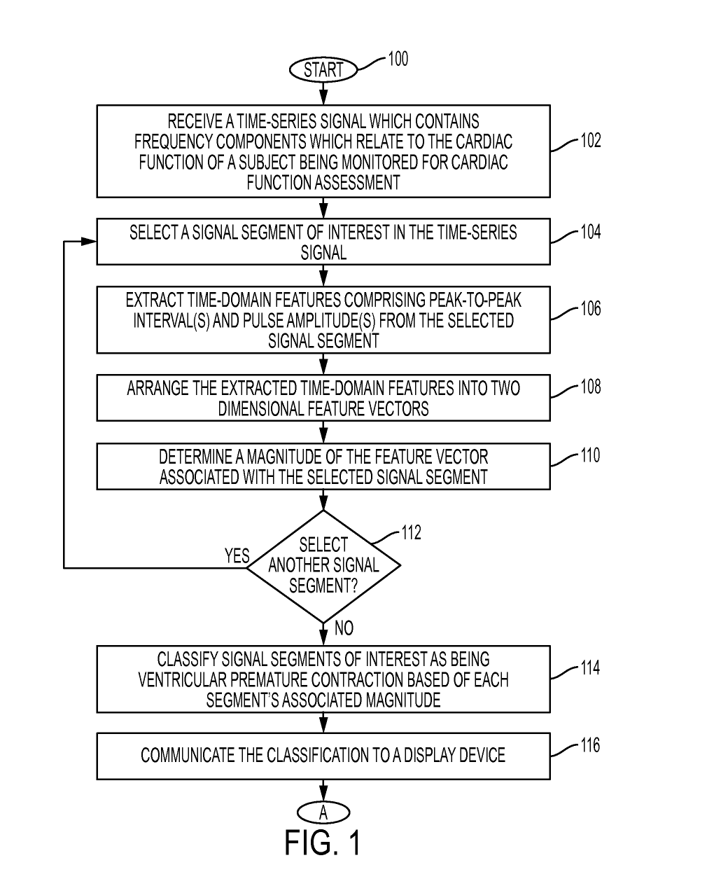

Reference is now being made to the flow diagram of FIG. 1 which illustrates one example embodiment of the present method for assessing patient risk for ventricular tachycardia in accordance with the methods disclosed herein. Flow processing begins at step 100 and immediately proceeds to step 102.

At step 102, receive a time-series signal containing frequency components which relate to a cardiac function of a subject being monitored for cardiac function assessment.

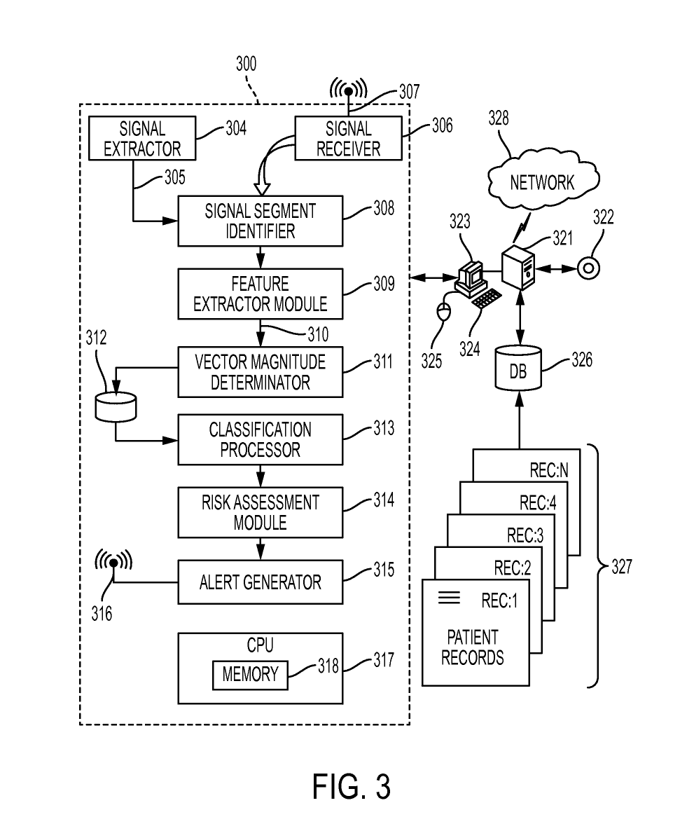

At step 104, select a signal segment of interest in the time-series signal. Signal segments have a fixed length. Such a selection may be effectuated by a user or technician using, for example, the workstation 321 of FIG. 3.

At step 106, extract time-domain features comprising peak-to-peak intervals between cardiac pulses and cardiac pulse amplitudes from the selected signal segment.

At step 108, arrange the extracted time-domain features into a two dimensional feature vector.

At step 110, determine a magnitude of the feature vector associated with the selected signal segment.

At step 112, a determination is made whether more signal segments are to be selected. If so then processing repeats with respect to step 104 wherein a next signal segment is identified or is otherwise selected for processing. Processing repeats in a similar manner until no more signal segments remain to be processed.

At step 114, classify signal segments as being ventricular premature contraction based on each segment's associated magnitude. Remaining signal segments are unclassified or are classified as normal sinus rhythm. In one embodiment, signal segments with a smallest magnitude are classified as being ventricular premature contraction.

At step 116, communicate the classification to a display device. One example display device is shown at 323 of FIG. 3. The classification can be communicated to a memory, a storage device, a handheld wireless device, a handheld cellular device, and/or a remote device over a network.

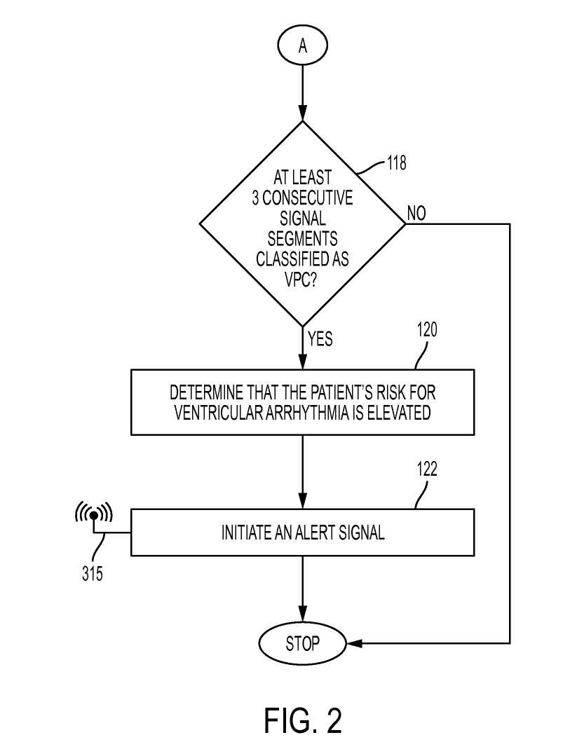

Reference is now being made to FIG. 2 which is a continuation of the flow diagram of FIG. 1 with flow processing continuing with respect to node A.

At step 118, a determination is made whether at least 3 consecutive signal segments have been classified as ventricular premature contraction. If not then, in this embodiment, further processing stop. Otherwise, processing continues with respect to step 120.

At step 120, determine that the subject's risk for ventricular tachycardia is elevated. Such a determination can be made by performing a manual count, or by automatic means.

At step 122, initiate an alert signal. The alert may take the form of a message displayed on a display device or a sound activated at, for example, a nurse's station or a display of a device. The alert may take the form of a colored or blinking light which provides a visible indication that an alert condition exists. The alert can be a text, audio, and/or video message. The alert signal may be communicated to one or more remote devices over a wired or wireless network. The alert may be sent directly to a handheld wireless cellular device of a medical professional. Thereafter, additional actions would be taken in response to the alert. In this embodiment, after the alert signal is initiated, further processing stops. In other embodiments, flow processing continues in a similar manner.

It should be appreciated that the flow diagrams depicted herein are illustrative. One or more of the operations in the flow diagrams may be performed in a differing order. Other operations may be added, modified, enhanced, or consolidated. Variations thereof are intended to fall within the scope of the appended claims.

### Block Diagram of Signal Processing System

Reference is now being made to FIG. 3 which illustrates a block diagram of one example signal processing system 300 for assessing patient risk for ventricular tachycardia as described with respect to the flow diagrams of FIGS. 1 and 2.

Signal Extractor 304 outputs a time-series signal 305. Signal Receiver 306, in the alternative, wirelessly receives a time-series signals via antenna 307. Signal Segment Identifier 308 receives the time-series signal from one or both of Signal Extractor 304 and Signal Receiver 306 and divides the time-series signal in signal segments of interest. The subject's cardiac specialist may facilitate such an identification using the workstation 321. Once signal segments of interest have been identified or otherwise selected, Feature Extractor Module 309 extracts, from each of the signal segments, time-domain features and outputs the extracted features in the form of a two-dimensional feature vector 310. Each feature vector is associated with a respective signal segment of interest. The feature vectors 310 are received by Magnitude Determinator 311 which determines a magnitude of each of the feature vectors. The results thereof are stored to storage device 312. Classification Processor 313 retrieves the magnitudes from the storage device and classifies signal segments of interest as ventricular premature contraction based on the determined magnitudes. Risk Assessment Module 314 determines whether at least three consecutive signal segments have been classified as being ventricular premature contraction. If so, then module 314 signals the Alert Generator 315 to initiate an alert via antenna 316. Central Processing Unit (CPU) 317 retrieves machine readable program instructions from Memory 318 and is provided to facilitate the functionality of any of the modules of the system 300. CPU 317, operating alone or in conjunction with other processors, may be configured to assist or otherwise perform the functionality of any of the modules or processing units of the system 300, as well as facilitating communication between the system 300 and the workstation 321.

Workstation 321 has a computer case which houses various components such as a motherboard with a processor and memory, a network card, a video card, a hard drive capable of reading/writing to machine readable media 322 such as a floppy disk, optical disk, CD-ROM, DVD, magnetic tape, and the like, and other software and hardware as is needed to perform the functionality of a computer workstation. The workstation includes a display device 323, such as a CRT, LCD, or touchscreen display, for displaying information, magnitudes, feature vectors, computed values, medical information, test results, and the like, which are produced or are otherwise generated by any of the modules or processing units of the system 300. A user can view any such information and make a selection from various menu options displayed thereon. Keyboard 324 and mouse 325 effectuate a user input or selection.

It should be appreciated that the workstation 321 has an operating system and other specialized software configured to display alphanumeric values, menus, scroll bars, dials, slideable bars, pull-down options, selectable buttons, and the like, for entering, selecting, modifying, and accepting information needed for performing various aspects of the methods disclosed herein. A user may use the workstation to identify signal segments of interest, set various parameters, and facilitate the functionality of any of the modules or processing units of the system 300. A user or technician may utilize the workstation to further modify the determined magnitudes of the feature vectors as is deemed appropriate. The user may adjust various parameters being utilized or dynamically adjust, in real-time, system or settings of any device used to capture the time-series signals. User inputs and selections may be stored/retrieved in any of the storage devices 312, 322 and 326. Default settings and initial parameters can be retrieved from any of the storage devices. The alert signal initiated by Alert Generator 314 may be received and viewed on the display device 323 of the workstation and/or communicated to one or more remote devices over network 328, which may utilize a wired, wireless, or cellular communication protocol.

The workstation implements a database in storage device 326 wherein patient records are stored, manipulated, and retrieved in response to a query. Such records, in various embodiments, take the form of patient medical history stored in association with information identifying the patient (collectively at 327). It should be appreciated that database 326 may be the same as storage device 312 or, if separate devices, may contain some or all of the information contained in either storage device. Although the database is shown as an external device, the database may be internal to the workstation mounted, for example, on a hard disk therein.

Although shown as a desktop computer, it should be appreciated that the workstation can be a laptop, mainframe, tablet, notebook, smartphone, or a special purpose computer such as an ASIC, or the like. The embodiment of the workstation is illustrative and may include other functionality known in the arts. Any of the components of the workstation may be placed in communication with any of the modules of system 300 or any devices placed in communication therewith. Moreover, any of the modules of system 300 can be placed in communication with storage device 326 and/or computer readable media 322 and may store/retrieve therefrom data, variables, records, parameters, functions, and/or machine readable/executable program instructions, as needed to perform their intended functionality. Further, any of the modules or processing units of the system 300 may be placed in communication with one or more remote devices over network 328. It should be appreciated that some or all of the functionality performed by any of the modules or processing units of system 300 can be performed, in whole or in part, by the workstation. The embodiment shown is illustrative and should not be viewed as limiting the scope of the appended claims strictly to that configuration. Various modules may designate one or more components which may, in turn, comprise software and/or hardware designed to perform the intended function.

The teachings hereof can be implemented in hardware or software using any known or later developed systems, structures, devices, and/or software by those skilled in the applicable arts without undue experimentation from the functional description provided herein with a general knowledge of the relevant arts. One or more aspects of the methods described herein are intended to be incorporated in an article of manufacture. The article of manufacture may be shipped, sold, leased, or otherwise provided separately either alone or as part of a product suite or a service. The above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are hereby incorporated by reference in their entirety.

What is claimed is:

1. A method for assessing risk for ventricular tachycardia from a time-series signal of a patient being monitored for cardiac function assessment, the method comprising:
receiving a time-series signal containing frequency components which relate to a cardiac function of a subject being monitored for cardiac function assessment;
identifying at least one signal segment of interest in said time-series signal, said signal segments having a fixed length;
extracting time-domain features from each of said identified signal segments of interest, said features comprising peak-to-peak interval between cardiac pulses and pulse amplitude, said extracted features being represented by at least one two dimensional feature vector, each of said feature vectors being associated with a respective signal segment;
determining a magnitude of each of said two dimensional feature vectors;
classifying signal segments of interest as being ventricular premature contraction based on a magnitude of said segment's respective associated feature vectors; and
in response to at least three consecutive signal segments being classified as being ventricular premature contraction, determining that said subject's risk of a ventricular tachycardia is elevated.

2. The method of claim 1, wherein said time-series signal is any of: a photoplethysmographic (PPG) signal, and a videoplethysmographic (VPG) signal.

3. The method of claim 1, wherein signal segments with a smallest magnitude are classified as being ventricular premature contraction.

4. The method of claim 1, wherein signal segments are classified using an unsupervised clustering method.

5. The method of claim 1, wherein, in advance of extracting said time-domain features, pre-processing said time-series signal to improve signal-to-noise ratio.

6. The method of claim 1, wherein, in advance of extracting said time-domain features, further comprising any of:
detrending said time-series signal to remove non-stationary components;
filtering said time-series signal to remove unwanted frequencies; and
smoothing said time-series signal to remove unwanted artifacts.

7. The method of claim 1, wherein, in advance of extracting said time-domain features, further comprising any of:
performing automatic peak detection on said signal segment to identify cardiac pulse peaks; and
filtering said signal segment to remove cardiac pulse peaks having more than at least at least a 20% change in consecutive peak-to-peak intervals.

8. The method of claim 1, wherein said signal segment is normalized to a frequency of a normalized heartbeat.

9. The method of claim 1, wherein a length of said signal segment comprises of any of: a single cardiac cycle, a normalized cardiac cycle, multiple cardiac cycles, and multiple normalized cardiac cycles.

10. The method of claim 1, wherein said three consecutive signal segments are within the same signal segment.

11. The method of claim 1, wherein, in response to any of said signal segments being classified as ventricular premature contraction, performing any of: initiating an alert, and signaling a medical professional.

12. The method of claim 1, further comprising communicating said classification to any of: a memory, a storage device, a display device, a handheld wireless device, a handheld cellular device, and a remote device over a network.

13. A system for assessing risk for ventricular tachycardia from a time-series signal of a patient being monitored for cardiac function assessment, the system comprising:
a memory; and
a processor in communication with said memory, said processor executing machine readable program instructions for performing:
receiving a time-series signal containing frequency components which relate to a cardiac function of a subject being monitored for cardiac function assessment;
identifying at least one signal segment of interest in said time-series signal, said signal segments having a fixed length;
extracting time-domain features from each of said identified signal segments of interest, said features comprising peak-to-peak interval between cardiac pulses and pulse amplitude, said extracted features being represented by at least one two dimensional feature vector, each of said feature vectors being associated with a respective signal segment;
determining a magnitude of each of said two dimensional feature vectors;
classifying signal segments of interest as being ventricular premature contraction based on a magnitude of said segment's respective associated feature vectors; and
in response to at least three consecutive signal segments being classified as being ventricular premature contraction, determining that said subject's risk of a ventricular tachycardia is elevated.

14. The system of claim 13, wherein said time-series signal is any of: a photoplethysmographic (PPG) signal, and a videoplethysmographic (VPG) signal.

15. The system of claim 13, wherein signal segments with a smallest magnitude are classified as being ventricular premature contraction.

16. The system of claim 13, wherein signal segments are classified using an unsupervised clustering method.

17. The system of claim 13, wherein, in advance of extracting said time-domain features, pre-processing said time-series signal to improve signal-to-noise ratio.

18. The system of claim 13, wherein, in advance of extracting said time-domain features, further comprising any of:
detrending said time-series signal to remove non-stationary components;
filtering said time-series signal to remove unwanted frequencies; and
smoothing said time-series signal to remove unwanted artifacts.

19. The system of claim 13, wherein, in advance of extracting said time-domain features, further comprising any of:
performing automatic peak detection on said signal segment to identify cardiac pulse peaks; and
filtering said signal segment to remove cardiac pulse peaks having more than at least at least a 20% change in consecutive peak-to-peak intervals.

20. The system of claim 13, wherein said signal segment is normalized to a frequency of a normalized heartbeat.

21. The system of claim 13, wherein a length of said signal segment comprises of any of: a single cardiac cycle, a normalized cardiac cycle, multiple cardiac cycles, and multiple normalized cardiac cycles.

22. The system of claim 1, wherein said three consecutive signal segments are within the same signal segment.

23. The system of claim 13, wherein, in response to any of said signal segments being classified as ventricular premature contraction, performing any of: initiating an alert, and signaling a medical professional.

24. The system of claim 13, further comprising communicating said classification to any of: a memory, a storage device, a display device, a handheld wireless device, a handheld cellular device, and a remote device over a network.
